(19) United States
(12) Patent Application Publication
(10) Pub. No.: US 2006/0235358 A1
(43) Pub. Date: Oct. 19, 2006

(54) Method and apparatus for tissue oxygenation

(76) Inventor: Jose Azocar, Hartford, CT (US)

Correspondence Address: HOLLAND & BONZAGNI, P.C., 171 DWIGHT ROAD, SUITE 302, LONGMEADOW, MA, 01106-1700, US

(21) Appl. No.: 11/405,970

(22) Filed: Apr. 17, 2006

Related U.S. Application Data
(60) Provisional application No. 60/672,913, filed on Apr. 18, 2005.

(51) Int. Cl. A61F 13/00

(52) U.S. Cl. 604/304; 602/48; 604/308

(57) ABSTRACT

Based on diffusion principles and Fick's Law of difflision, Applicant has developed a method and two apparatuses to increase tissue oxygenation through a patient's damaged tissue to heal wounds quickly. Applicant's method is to create an enclosed environment, immediately outside a patient's damaged tissue, in which the oxygen concentration is reduced to prompt an outward supply of oxygen flows from blood capillaries to the damaged tissue and eventually out of the skin. Applicants' disclosed apparatuses use two different ways to reduce the oxygen: oxygen scavengers (e.g., oxygen-absorbing packets); and burning off the oxygen.

### RELATED APPLICATION

This application claims priority from U.S. Provisional Patent Application Ser. No. 60/672,913, filed Apr. 18, 2005. Applicant hereby incorporates the disclosure of that application by reference.

### FIELD OF INVENTION

This invention relates to medical devices and processes for treating a human patient's damaged tissue (e.g., wounds) through tissue oxygenation.

### BACKGROUND OF THE INVENTION

Oxygen has a basic role in wound healing. It is essential in tissue repair due to any injuries: chemical, biological (infections) and physical (e.g., UV light, fire). If the tissue is damaged, it usually disrupts the normal capillary network, reducing the amount of oxygen being supplied. Damaged tissue also has an increase in metabolic rate and demand for oxygen to repair itself. With extra oxygen supply the damaged tissue can overcome these obstacles and increase the tissue repair rate.

On the other hand, some diseases such as diabetes and hypertension may damage the vascular system affecting circulation. As a consequence, enhancement of tissue oxygenation is a primary goal in therapy of lesions as in the case of tissue infections and ulcers.

Since human skin is permeable and allows small non-polar molecules such as oxygen to readily dissolve in its lipid bi-layers, it is able to exchange gasses like oxygen and carbon dioxide with the atmosphere. Under normal conditions most of the oxygen supply to the outermost layer of the skin up to a depth of 0.40 mm comes from atmospheric O₂ rather than being supplied from the blood capillaries.

Earlier methods, such as Hyperbaric Oxygen Therapy, focus on outward oxygen diffusion from the capillary network towards the skin. Applicant is proposing a technique of promoting oxygen diffusion from the capillary network towards the subcutaneous tissue and skin under normobaric, rather than hyperbaric, conditions.

Accordingly, the primary object of the present invention is to create an enclosed environment, immediately outside a patient's damaged skin, in which the oxygen concentration is reduced to prompt an outward supply of oxygen flows from blood capillaries to the damaged tissue and eventually out of the skin.

Other objects and advantages of the current invention will become more readily apparent when the following written description is read in conjunction with the accompanying drawings.

### BRIEF DESCRIPTION OF THE DRAWINGS

FIG. 1 shows a preferred apparatus, constructed in accordance with the present invention, for treating damaged tissue by increasing tissue oxygenation;

FIG. 2 shows another preferred apparatus, constructed in accordance with the present invention, for treating damaged tissue by increasing tissue oxygenation;

FIG. 3 shows a tiered cylinder, with oxygen scavengers, used in a preliminary study of efficacy of the invention; and

FIG. 4 is a chart showing the test results from the preliminary study.

### SUMMARY OF INVENTION

Two alternate apparatuses, and a method, are disclosed for treating damaged tissue (e.g., wounds) by increasing tissue oxygenation. One preferred apparatus involves an oxygen-absorbing multi-layered material, preferably in contact with the skin, which creates a sealed normobaric environment over an area of damaged tissue being treated. The oxygen-absorbing material reduces the concentration of oxygen inside that environment. The other preferred apparatus bums oxygen inside a closed environment to create an oxygen deprived environment that surrounds the tissue being treated. Reducing the concentration of oxygen prompts an outward supply of oxygen flows from blood capillaries to the damaged tissue and eventually out of the skin.

### DESCRIPTION OF THE PREFERRED EMBODIMENTS

Supplying additional amounts of oxygen to skin tissue in need of an increase oxygen for therapeutic purposes, such as in the case of ulcers, wounds, or similar situations where the circulation (and source of oxygen) is compromised (such as in peripheral vascular disease), is essential to tissue healing. The present invention, nicknamed the “Tissue Oxygenator,” when used on a patient, causes a constant outward flow of oxygen supply from blood capillaries to the tissue and eventually out of the skin. The Tissue Oxygenator is applied on the outside of a target area where for therapeutic purposes, the concentration or flow of oxygen needs to be promoted and/or increased. The Tissue Oxygenator involves a closed, normobaric environment in which oxygen is burnt off, or absorbed by an oxygen-absorbing material, to reduce the concentration levels of O₂ inside the Oxygenator. Through basic principles and laws of diffusion, the lower concentration levels of O₂ created by the oxygen-absorbing material (i.e., oxygen scavengers) prompts an outward oxygen flow from the blood capillaries through tissue and into the Tissue Oxygenator.

In a preferred embodiment (see FIG. 1), the Tissue Oxygenator involves a closed, dome-like, normobaric environment or chamber formed by a bandage 10. The bandage is made of oxygen-absorbing, multi-layer sheets such as those disclosed in U.S. Pat. No. 5,089,323 to Nakae et al; U.S. Pat. No. 6,063,503 to Hatakeyama et al.; U.S. Pat. No. 6,391,407 to Kashiba et al.; U.S. Pat. No. 6,503,587 to Kashiba et al.; and U.S. Pat. No. 6,746,772 to Kashiba et al. These patents disclose oxygen-absorbing, multi-layer sheets used in packages for foods, pharmaceuticals or flowers. There is no mention of using any of these prior patented sheets, though, as bandages over damaged human tissue in an enclosed environment; further, no mention of utilizing them to oxygenate the tissue.

U.S. Pat. No. 5,089,323 to Nakae et al. discloses an oxygen-absorbing sheet. The sheet includes a thermoplastic resin and an oxygen absorbent made of iron powder.

U.S. Pat. No. 6,063,503 to Hatakeyama et al. discloses an oxygen-absorbing multi-layer film. The film includes an oxygen permeating layer; a deoxidizing resin layer containing iron powder; a smoothing layer; and a gas barrier layer.

U.S. Pat. No. 6,391,407 to Kashiba et al. discloses an oxygen-absorbing multi-layer film. Moving from the “outside in,” the film includes two contiguous oxygen-permeating layers 11, 12; an oxygen-absorbing layer containing an iron deoxidizing agent 2; a gas barrier layer 3; and an oxygen-permeating layer 4.

U.S. Pat. No. 6,503,587 to Kashiba et aL discloses an oxygen-absorbing multi-layer film suitable for preserving food. Referring to FIG. 1 of that patent, the multi-layer film comprises an outer layer 1 to which pigment is added; a contiguous thermoplastic resin layer 3; a gas barrier layer 4; and a protecting layer 5. Layers 3, 4, 5 are successively laminated on layer 1. Particles of an oxygen-absorbing agent 2 (preferably, iron powder) are locally interspersed between layers 1 and 3, with some of the particles being distributed in the interface between the adjacent layers (1 and 3) while some particles are present in one of the layers (1 or 3) and the other particles are present in both the layers (1 and 3). The disclosure of U.S. Pat. No. 6,503,587 is hereby incorporated by reference.

U.S. Pat. No. 6,746,772 to Kashiba et al. discloses an oxygen-absorbing multi-layer film. The film comprises an outer layer made of a thermoplastic resin; an adhesive layer comprising an epoxy resin; and an oxygen-absorbing layer, made of a thermoplastic resin, with an iron based oxygen-absorbing agent incorporated therein.

Applicant's preferred method and apparatus (see FIG. 1 in the present application) utilize an oxygen-absorbing material such as the one disclosed in U.S. Pat. No. 6,503,587. A multi-layered bandage can be ergonomically shaped to fit the contours of the arm, leg, or other body part being treated. It surrounds the area of the body tissue being treated and creates an enclosed, normobaric environment that is bordered by the skin and the oxygen-absorbing material separating it from the atmosphere.

Referring to FIG. 1, Applicant's preferred bandage 10 comprises a multi-layered oxygen-absorbing material having a structure similar to FIG. 1 of U.S. Pat. No. 6,503,587 (described above). Moving from the outside in, Applicant's bandage 10 comprises: an impermeable casing layer 12 of thermoplastic resin; an oxygen-absorbing layer 14 of thermoplastic resin having an iron based oxygen-absorbing agent 16 (e.g., iron powder); a gas barrier layer 18 made of an oxygen permeable thermoplastic resin; and, a penultimate layer 20 also made of thermoplastic resin. In addition; Applicant's bandage 10 includes an additional, adhesive layer 22 (a.k.a. attachment means) to stick the bandage onto the patient's skin 24 so that an enclosed dome-like chamber (a.k.a. environment) is sealed on top of damaged tissue 26.

Like all gas molecules, oxygen diffuses down its concentration gradient from a space of higher amounts of oxygen molecules to a space of lesser amounts of oxygen molecules in order to reach dynamic equilibrium. Lowering the concentration of oxygen inside the chamber of the Tissue Oxygenator by the oxygen-binding material, will promote a gradient of oxygen that will flow from the capillaries trough the skin towards the chamber where the oxygen concentration is lower. The result is an increase number of oxygen molecules traveling through the target area (see FIG. 1) and promoting the oxygen-dependent healing process.

Since human skin is permeable and allows small non-polar molecules such as oxygen to readily dissolve in its lipid bi-layers, the skin is able to exchange gasses like oxygen and carbon dioxide with the atmosphere. Under normal conditions most of the oxygen supply to the outermost layer of the skin up to a depth of 0.40 mm comes from atmospheric O₂ rather than being supplied from the blood capillaries. Oxygen diffusion was also found to be more facilitated when the skin was moist.

Fick's First Law of Diffusion (“Fick's Law”) describes the passive movement of molecules down its concentration gradient. If this law is applied to the finction of the Tissue Oxygenator, Applicant believes that it proves that the Oxygenator works properly.

The following equation, based upon Fick's law, gives the steady state relationship for the rate of oxygen transfer (“mass transfer”):

$$W = \frac{( {C}_{1} - {C}_{2} ) ( A ) ( D )}{L}$$

where:
W=mass transfer rate;
C₁=higher concentration of oxygen in the capillaries;
C₂ =lower concentration of oxygen above the tissue;
A=tissue area across which diffusion occurs;
D=diffusion or permeability coefficient; and
L=thickness of the damaged tissue (a.k.a. length of the diffusion path).

Using this formula, the flux of oxygen and the direction of its diffusion can be calculated. Since the concentration of O₂ is greater outside the skin, the oxygen flux usually transverses a path from outside to inside the skin. The oxygen flux follows the path of least resistance and seeks to balance the pressures.

The ability for oxygen to diffuse from the capillaries through tissue 26 and eventually out of the body (skin 24) can only occur if certain conditions are established for Fick's Law of diffusion and basic properties of diffusion to apply. To meet these conditions, the concentration of oxygen must be lower outside the body than inside of it, which is the function of the Tissue Oxygenator. In addition, human skin allows rapid diffusion of small non-polar molecules such as oxygen. Under Fick's Law, if all the properties are upheld, then oxygen will diffuse from the capillaries towards the skin down its concentration gradient to an oxygen depleted environment-supplying tissue with much needed oxygen.

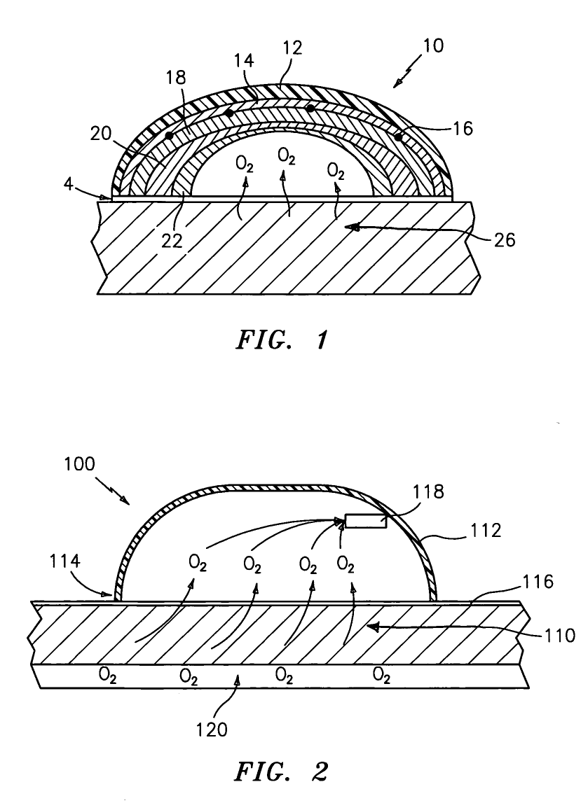

A second, preferred embodiment of the “Tissue Oxygenator” is shown in Applicant's FIG. 2. In that embodiment, another bandage 100 creates an enclosed dome-like environment on top of damaged tissue 110. The bandage comprises an impermeable casing layer 112 of thermoplastic resin impregnated with pigment; and, an adhesive layer 114 to stick the bandage onto the patient's skin 116 so that an enclosed dome-like chamber (a.k.a. environment) is sealed. The bandage also includes any suitable burning means, such as ignition system 118 (e.g., a sterile electrical wire) or other “oxygen depletion means,” exposed inside the dome. Igniter 118 is designed to burn off oxygen inside the dome to create an oxygen deprived environment. Reducing the concentration of oxygen prompts an outward supply of oxygen flows from blood capillaries 120 to the damaged tissue 100 and eventually out of the skin 114.

Other portions of bandage 10 could be incorporated into the bandage 100, such as a gas barrier layer (not shown in FIG. 2).

It should be understood that other, obvious structural modifications can be made without departing from the. spirit or scope of the invention. For example, instead of a soft bandage, a plastic or glass container could be used on the surface of the skin with a wound or ulceration. Inside the container would be an oxygen scavenger (e.g., iron powder), or an igniter, to cause the oxygen deprived environment. Accordingly, reference should be made to the accompanying Claims, rather then the foregoing description, to determine the scope of the invention.

### Preliminary Study for Efficacy

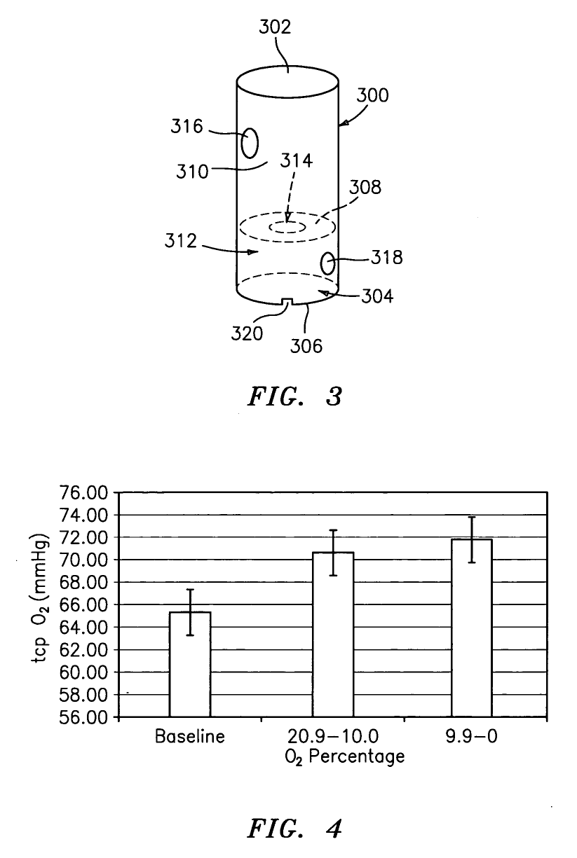

To create the desired experimental enclosed environment a hollow, tiered glass cylinder 300 was designed with a height of 16.20 cm, diameter of 6.00 cm, a thickness of 0.35 cm, and a volume of 457.812 cm³ (see FIG. 3). The cylinder had an integral top 302 but was bottomless. The cylinder's bottom opening 304 was designed to overlie a patient's damaged skin (not shown), with the cylinder's bottom edge or rim 306 contacting the patient.

The cylinder was designed to contain oxygen scavengers (not shown) to reduce the oxygen concentration within the cylinder, but prevent skin contact with the oxygen scavengers. To achieve this, a ring-shaped divider 308 was attached to the cylinder to create upper 310 and lower 312 compartments. Oxygen scavengers (not shown) were located in the upper compartment. A hole 314 in the ring divider 308 was small enough so that the scavengers would not fall through, but large enough that the scavengers would be able to absorb oxygen from the lower compartment 312. Two circular openings 316, 318 with diameters of 2.90 cm were located at each compartment 310, 312. The upper opening 316 allowed for the placement of the oxygen scavengers and was later sealed with a rubber cap (not shown) to prevent any air exchange with the atmosphere. An Oxygen Analyzer probe (not shown), which measured the concentration of oxygen, fit the lower opening 318. A small crevice 320 at the bottom edge of the glass allowed an electrode probe that measured the transcutaneous partial pressure of oxygen (tcpO₂) of the skin within the enclosed, normobaric environment.

To reduce the oxygen concentration within the enclosed environment Applicant used oxygen absorbing packets (“Freshpack” by Multisorb Technologies, Inc., Buffalo, N.Y.) containing the oxygen scavenger ferrous sulfate. Other packets could easily have been used.

To determine the changes of oxygen concentration within the enclosed environment, Applicant used a standard Oxygen Analyzer (not shown) (manufactured by Vascular Technology, Inc., Lowell, Mass.), which measured the percentage of oxygen concentration. Transcutaneous oxygen partial pressure (tcpO₂) was measured with a TCM 3 TINA equipment (manufactured by Radiometer Medical ApS, Copenhagen, Demark), which mapped actual oxygen supply available for the skin cells in the desired area.

Measurement of tcpO2 was done as described by the manufacturer. Briefly, three subjects were placed sitting in a chair with their leg at a right angle (i.e., 90°). A quadriceps area of the leg was shaved and then cleaned with rubbing alcohol and the tcpO₂ electrode was placed over the cleaned area of the quadriceps. The oxygen eliminating device was then placed on top of the electrode, so the TCM would measure the tcpO₂ in the skin covered by the enclosed environment. Gel was put around the borders of the enclosed environment to avoid air from entering or exiting the device. The subjects then rested for 40 minutes, minimizing movement as much as possible, to establish a baseline. After a baseline was reached the oxygen scavenger packets were then inserted in the device. Measurements of the tcpO₂ and O₂ concentration were recorded every minute. This procedure was done several times in order to standardize the technique and determine the reproducibility of the results. A total of 11 sets experiments were carried as described above. The results are shown in chart form in FIG. 4.

As shown in FIG. 4, the skin tcpO₂ (i.e., represented by the “Y” axis) increased as the percentage of oxygen (i.e., represented by the “X” axis) decreased within the enclosed environment. Standard error of mean of the tcpO₂ was then calculated at the baseline and at oxygen concentration of: 20.9 to 10%; and between 9.99 and 0%. In our experiments, the mean tcpO₂ at the baseline was significantly lower than the tcpO₂ obtained when the concentration of oxygen in the enclosed environment immediately outside the skin was lowered. The greatest increase in the tcpO₂ was reached at oxygen concentrations close to zero within the enclosed environment.

Since tcpO2 maps actual oxygen supply available for the skin tissue cells, an increase in tcpO₂ indicates an increase oxygen supply to the skin and subcutaneous tissue. TcpO₂ predicts with high accuracy the healing of chronic wounds when hyperbaric oxygen therapy is used (10). Applicant believes that the test results indicate that the technique described here, by increasing cutaneous oxygenation, should promote wound healing.

I claim:

1. A method of treating a human patient's damaged tissue comprising:
a. creating an enclosed, normobaric environment over a particular body part being treated having the damaged tissue, which includes the sub-steps of:
i. ergonomically shaping a bandage to fit the contours of that body part; and,
ii. sealing off the area of the tissue being treated with the bandage; and,
b. reducing the level of oxygen within the enclosed environment to prompt an outward supply of oxygen flows from capillaries to the damaged tissue and eventually out of the body part.

2. A method of treating wounds comprising:
a. creating an enclosed normobaric environment over a patient's damaged tissue; and
b. reducing the level of oxygen within the enclosed environment to prompt an outward supply of oxygen flow from blood capillaries to the damaged tissue and eventually out of skin of the patient.

3. The method of claim 2 wherein the step of creating the enclosed environment comprises securing a bandage to the patient to create the enclosed environment.

4. The method of claim 2 wherein the step of reducing the oxygen concentration comprises burning off oxygen within the enclosed environment.

5. The method of claim 2 wherein the step of reducing the oxygen concentration comprises using at least one oxygen scavenger within the enclosed environment but away from the patient's skin.

6. Apparatus for treating a patient's damaged tissue comprising:
a. a multi-layered bandage having a dome-like structure, wherein the bandage comprises:
i. an impermeable casing layer;
ii. a contiguous oxygen-absorbing layer having an oxygen-absorbing agent;
iii. a gas barrier layer contiguous to the oxygen-absorbing layer; and,
iv. attachment means to removably attach the bandage onto skin of the patient so that an enclosed domelike envirorunent is sealed on top of damaged tissue.

7. The apparatus of claim 6 wherein the oxygen-absorbing agent comprises iron powder.

8. The apparatus of claim 6 wherein the oxygen-absorbing agent is a oxygen-absorbing packet.

9. Apparatus for treating a patient's damaged tissue comprising:
a. a bandage adapted in size and shape to fit over the damaged tissue, wherein the bandage comprises:
i. an impermeable casing layer; and
ii. attachment means to removably attach the bandage onto skin of the patient so that an enclosed, domelike, normobaric environment is sealed on top of damaged tissue; and
b. oxygen depletion means for depleting oxygen inside the enclosed environment.
